(19) United States
(12) Patent Application Publication
(10) Pub. No.: US 2002/0132792 A1
(43) Pub. Date: Sep. 19, 2002

(54) Aryl-substituted indirubin derivatives, their production and use

(75) Inventors: Olaf Prien, Berlin (DE); Andreas Steinmeyer, Berlin (DE); Gerhard Siemeister, Berlin (DE); Rolf Jautelat, Berlin (DE)

Correspondence Address: MILLEN, WHITE, ZELANO & BRANIGAN, P.C., 2200 CLARENDON BLVD., SUITE 1400, ARLINGTON, VA, 22201, US

(73) Assignee: Schering AG, Berlin (DE)

(21) Appl. No.: 09/995,862

(22) Filed: Nov. 29, 2001

(30) Foreign Application Priority Data
Nov. 30, 2000 (DE) 10061162.1

(51) Int. Cl. A61K 31/695; C 07D 4 3/02; A61K 31/404

(52) U.S. Cl. 514/63; 514/411; 514/414; 548/408; 548/427; 548/494

(57) ABSTRACT

Aryl-substituted indirubin derivatives, their production and their intermediate products for production, as well as their use as medications for treating cancer, such as solid tumors and leukemia; auto-immune diseases, such as psoriasis, alopecia and multiple sclerosis, chemotherapy agent-induced alopecia and mucositis; cardiovascular diseases, such as stenoses, arterioscleroses and restenoses; infectious diseases, such as, e.g., those caused by unicellular parasites, such as trypanosoma, toxoplasma or plasmodium, or those caused by fungi; nephrological diseases, such as, e.g., glomerulonephritis; chronic neurodegenerative diseases, such as Huntington's disease, amyotrophic lateral sclerosis, Parkinson's disease, AIDS dementia and Alzheimer's disease; acute neurodegenerative diseases, such as ischemias of the brain and neurotraumas; viral infections, such as, e.g., cytomegalic infections, herpes, hepatitis B and C, and HIV diseases, are described.

This invention relates to aryl-substituted indirubin derivatives, their production and their use as medications for treating various diseases.

It is known from traditional Chinese medicine that indirubin and several indirubin derivatives are effective against certain forms of cancer. In addition to antineoplastic actions, indirubin-3′-oxime-methyl ether and indirubin-3′-oxime-ethyl ether also exhibit an in vitro inhibiting action on various leukemia cell lines of patients with acute lymphatic, acute myeloid and chronic granulocytic leukemia (Li et al., 1996, Bull. Chem. Soc. Japan, 69, 1621-1627 and Tian et al., 1995, Chemical Research in Chinese Universities, 11, 75-78).

As early as 1913, a patent on the production of alkyl ethers of indirubin oximes was granted by the German Imperial Patent Office (No. 282278).

The synthesis of selected indirubin derivatives, as well as their property as active active ingredients for treating cancer, thus for example as a preparation of the natural cocktail “Dang Gui Lu Hui Wan,” is described in Chinese J. Intern. Med. 15, 86-88, (1979).

Basic works for the synthesis of indirubin and indirubin derivatives are described in G. A. Russell, G. Kaupp, J. Am. Chem. Soc. 1969, 91, 3851-3859.

The pharmacological action of several indirubin derivatives is described in WO 99/62503. In addition to a large number of other indirubins, aryl-substituted indirubin derivatives are also mentioned there quite generally. A process for the production of aryl-substituted indirubin derivatives as well as their effectiveness is not shown, however. The description contains only examples with structurally remote indirubin derivatives, their production and use. Also, what compounds are now the truly effective and selective compounds is not disclosed in WO 99/62503.

Based on the advantageous properties of the compound class, a great need for more selective and especially more effective indirubin derivatives for treating various diseases continues to exist. This includes, for example, cancer, such as solid tumors and leukemia; auto-immune diseases, such as psoriasis, alopecia and multiple sclerosis, chemotherapy agent-induced alopecia and mucositis; cardiovascular diseases, such as stenoses, arterioscleroses and restenoses; infectious diseases, such as, e.g., those caused by unicellular parasites, such as trypanosoma, toxoplasma or plasmodium, or those caused by fungi; nephrological diseases, such as, e.g., glomerulonephritis; chronic neurodegenerative diseases, such as Huntington's disease, amyotrophic lateral sclerosis, Parkinson's disease, AIDS dementia and Alzheimer's disease; acute neurodegenerative diseases, such as ischemias of the brain and neurotraumas; viral infections, such as, e.g., cytomegalic infections, herpes, hepatitis B and C, and HIV diseases.

It is therefore desirable to obtain the compounds from the considerable number of indirubin derivatives that also have the above-mentioned advantages.

It has now been found that aryl-substituted indirubin derivatives of general formula I,

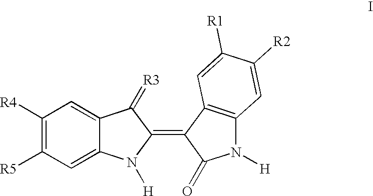

in which

R¹, R², R⁴ and R⁵ stand for hydrogen, hydroxy, C₁₋₆ alkoxy, aryloxy, trifluoromethyl, nitro, halogen, cyano or the group —SOₚR⁶, —SO₂NR⁷R⁸, or —COR⁹; or for aryl or heteroaryl that is optionally substituted by the same or a different component in one or more places with hydroxy, halogen, C₁-C₆ alkyl, amino, nitro, trifluoromethyl, —O—Si(C₁-C₆ alkyl)₃, cyano, COOC₁₋₄ alkyl, CO₁₋₄ alkyl, CO₁₋₄ aryl, SO₂(C₁₋₄ alkyl), SO₂ aryl and/or C₁-C₆ alkoxy,

R³ stands for oxygen or the group —NOR⁹, or

R¹ and R² or

R⁴ and R⁵ optionally form another C₃-C₆-membered ring,

R⁶ stands for hydrogen, for C₁-C₁₈ alkyl that is optionally substituted in one or more places with halogen, hydroxy and/or amino; aryl, heteroaryl or C₃₋₈ cycloalkyl that is optionally substituted in one or more places with halogen, hydroxy, amino, C₁-C₅ alkyl and/or C₁-C₆ alkoxy,

R7 and R8 are the same or different and stand for hydrogen or for C₁-C₁₈ alkyl, aryl, heteroaryl or acyl that is optionally substituted in one or more places with hydroxy, halogen and/or amino; or C₁₋₁₈ alkyl, C₃₋₈ cycloalkyl or C₃₋₈ cycloalkenyl that is optionally interrupted by one or more oxygen atoms, or

R⁷ or R⁸ together with the nitrogen atom of the amino group forms a C₃₋₈ cycloalkyl, which can contain one or more additional heteroatoms,

R⁹ stands for hydrogen or for C₁₋₆ alkyl that is optionally substituted in one or more places with hydroxy, halogen, C₁₋₄ alkoxy, amino, nitro, trifluoromethyl, cyano, COOC₁₋₄ alkyl, CO₁₋₄ alkyl, CO₁₋₄ aryl, SO₂(C₁₋₄ alkyl) and/or SO₂-aryl; or for aryl or heteroaryl that is optionally substituted in one or more places with halogen, C₁-C₆ alkyl, hydroxy, amino, nitro, trifluoromethyl, cyano, COOC₁₋₄ alkyl, CO₁₋₄ alkyl, CO₁₋₄ aryl, SO₂(C₁₋₄ alkyl), SO₂ arylamino and/or C₁-C₆ alkoxy, and

p is 0, 1 or 2,

whereby at least one of the two radicals R¹ and R² or radicals R4 and R⁵ means the aryl or hetaryl radical, as well as isomers and salts thereof, exhibit a surprising and, moreover, significantly better action on the isolated enzyme and on the cell compared to the known indirubin derivatives.

The compounds of general formula I also contain the possible tautomeric forms and comprise the E- or Z-isomers or, if a chiral center is present, the racemates or enantiomers.

Alkyl is defined in each case as a straight-chain or branched alkyl radical, such as, for example, methyl, ethyl, propyl, isopropyl, butyl, isobutyl, sec-butyl, tert-butyl, pentyl, hexyl, whereby C₁₋₄ alkyl radicals are preferred.

Halogen is defined in each case as fluorine, chlorine, bromine or iodine.

In each case, the aryl radical has 6-12 carbon atoms, such as, for example, naphthyl, biphenyl and especially phenyl.

In each case, the heteroaryl radical can be benzocondensed. For example, thiophene, furan, oxazole, thiazole, imidazole and benzo derivatives thereof can be mentioned as 5-ring heteroaromatic compounds, and pyridine, pyrimidine, triazine, quinoline, isoquinoline and benzo derivatives thereof can be mentioned as 6-ring heteroaromatic compounds.

If an acid group is included, the physiologically compatible salts of organic and inorganic bases are suitable as salts, such as, for example, the readily soluble alkali salts and alkaline-earth salts as well as N-methyl-glucamine, dimethyl-glucamine, ethyl-glucamine, lysine, 1,6-hexadiamine, ethanolamine, glucosamine, sarcosine, serinol, tris-hydroxy-methyl-amino-methane, aminopropanediol, Sovak base, and 1-amino-2,3,4-butanetriol.

If a basic group is included, the physiologically compatible salts of organic and inorganic acids are suitable, such as hydrochloric acid, sulfuric acid, phosphoric acid, citric acid, tartaric acid, i.a.

Especially effective are those compounds of general formula I, in which

R¹ stands for aryl or heteroaryl that is optionally substituted by the same or a different component in one or more places with halogen, hydroxy, C₁-C₆ alkyl, amino, nitro, trifluoromethyl, —O—Si(C₁-C alkyl)₃, cyano, COOC₁₋₄ alkyl, CO₁₋₄ alkyl, CO₁₋₄ aryl, SO₂(C₁₋₄ alkyl), SO₂ aryl and/or C₁-C₆ alkoxy, and

R² stands for hydrogen, hydroxy, C₁₋₆ alkoxy, aryloxy, trifluoromethyl, nitro, halogen, cyano or the group —SOₚR⁶, —SO₂NR⁷R⁸, or —COR⁹, or

R¹ stands for hydrogen, hydroxy, C₁₋₆ alkoxy, aryloxy, trifluoromethyl, nitro, halogen, cyano or the group —SOₚR⁶, —SO₂NR⁷R⁸, or —COR⁹, and

R² stands for aryl or heteroaryl that is optionally substituted in one or more places with halogen, hydroxy, C₁-C₆ alkyl, amino, nitro, trifluoromethyl, cyano, COOC₁₋₄ alkyl, CO₁₋₄ alkyl, CO₁₋₄ aryl, SO₂(C₁₋₄ alkyl), SO₂ aryl and/or C₁-C₆ alkoxy,

R⁴ and R⁵ stand for hydrogen, hydroxy, C₁₋₆ alkoxy, aryloxy, trifluoromethyl, nitro, halogen, cyano or the group —SOₚR⁶, —SO₂NR⁷R⁸, or —COR⁹; or for aryl or heteroaryl that is optionally substituted in one or more places with halogen, hydroxy, C₁-C₆ alkyl, amino, nitro, trifluoromethyl, cyano, COOC₁₋₄ alkyl, CO₁₋₄ alkyl, CO₁₋₄ aryl, SO₂(C₁₋₄ alkyl), SO₂ aryl and/or C₁-C₆ alkoxy,

R³ stands for oxygen or the group —NOR⁹, or R¹ and R² or

R⁴ and R⁵ optionally form another C₃-C₆-membered ring,

R6 stands for hydrogen or C₁₋₁₈ alkyl,

R⁷ and R8 are the same or different and stand for hydrogen or for C₁₋₁₈ alkyl, aryl, heteroaryl or acyl that is optionally substituted in one or more places with hydroxy, halogen and/or amino; or C₁₋₁₈ alkyl, C₃₋₈ cycloalkyl or C₃₋₈ cycloalkenyl that is optionally interrupted by one or more oxygen atoms, or

R⁷ or R8 together with the nitrogen atom of the amino group forms a C cycloalkyl, which can contain one or more other heteroatoms,

R⁹ stands for hydrogen or for C₁₋₆ alkyl that is optionally substituted in one or more places with hydroxy, halogen, C₁₋₄ alkoxy, amino, nitro, trifluoromethyl, cyano, COOC₁₋₄ alkyl, CO₁₋₄ alkyl, CO₁₋₄ aryl, SO₂(C₁₋₄ alkyl) and/or SO₂-aryl; or for aryl or heteroaryl that is optionally substituted in one or more places with halogen, C₁-C₆ alkyl, hydroxy, amino, nitro, trifluoromethyl, cyano, COOC₁₋₄ alkyl, CO₁₋₄ alkyl, CO₁₋₄ aryl, SO₂(C₁₋₄ alkyl), SO₂ arylamino and/or C₁-C₆ alkoxy, and

p is 0, 1 or 2, as well as isomers and salts thereof.

Those compounds of general formula I have proven quite especially effective in which

R¹ stands for phenyl, thiophenyl, furanyl, oxazolyl, thiazolyl, imidazolyl, pyridinyl, pyrimidinyl, triazinyl, quinolinyl or isoquinolinyl that is optionally substituted by the same or a different component in one or more places with halogen, hydroxy, C₁-C₆ alkyl, amino, nitro, trifluoromethyl, —O—Si(C₁-C₆ alkyl)₃, cyano, COOC₁₋₄ alkyl, CO₁₋₄ alkyl, CO₁₋₄ aryl, SO₂(C₁₋₄ alkyl), SO₂ aryl and/or C₁-C₆ alkoxy, and

R² stands for hydrogen, hydroxy, C₁₋₆ alkoxy, aryloxy, trifluoromethyl, nitro, halogen, cyano or the group —SOₚR⁶ —SO₂NR⁷R⁸, or —COR⁹, or

R¹ stands for hydrogen, hydroxy, C₁₋₆ alkoxy, aryloxy, —CF₃, nitro, halogen, cyano or the group —SOₚR⁶, —SO₂NR⁷R⁸, or —COR⁹, and

R² stands for phenyl, thiophenyl, furanyl, oxazolyl, thiazolyl, imidazolyl, pyridinyl, pyrimidinyl, triazinyl, quinolinyl or isoquinolinyl that is optionally substituted in one or more places with halogen, hydroxy, C₁-C₆ alkyl, amino, nitro, trifluoromethyl, cyano, COOC₁₋₄ alkyl, CO₁₋₄ alkyl, CO₁₋₄ aryl, SO₂(C₁₋₄ alkyl), SO₂ aryl and/or C₁₋₆ alkoxy, R⁴ and R⁵ stand for hydrogen, hydroxy, C₁₋₆ alkoxy, aryloxy, —CF₃, nitro, halogen, cyano or the group —SOₚR⁶, —SO₂NR⁷R⁸, or —COR⁹; or for aryl or heteroaryl that is optionally substituted in one or more places with halogen, hydroxy, C₁-C₆ alkyl, amino, nitro, trifluoromethyl, cyano, COOC₁₋₄ alkyl, CO₁₋₄ alkyl, CO₁₋₄ aryl, SO₂(C₁₋₄ alkyl), SO₂ aryl and/or C₁-C₆ alkoxy,

R³ stands for oxygen or the group —NOR⁹, or

R¹ and R² or

R⁴ and R⁵ optionally form another C₃-C₆-membered ring,

R⁶ stands for hydrogen or C₁₋₁₈ alkyl,

R⁷ and R⁸ are the same or different and stand for hydrogen or for C₁₋₁₈ alkyl, aryl, heteroaryl or acyl that is optionally substituted in one or more places with hydroxy, halogen and/or amino; or C₁₋₁₈ alkyl, C₃₋₈ cycloalkyl or C₃₋₈ cycloalkenyl that is optionally interrupted by one or more oxygen atoms, or

R⁷ or R8 together with the nitrogen atom of the amino group forms a C₃₋₈ cycloalkyl, which can contain one or more other heteroatoms,

R⁹ stands for hydrogen or for C₁₋₆ alkyl that is optionally substituted in one or more places with hydroxy, halogen, C₁₋₄ alkoxy, amino, nitro, trifluoromethyl, cyano, COOC₁₋₄ alkyl, CO₁₋₄ alkyl, CO ₁₄ aryl, SO₂(C₁₋₄ alkyl) and/or SO₂ aryl; or for aryl or heteroaryl that is optionally substituted in one or more places with halogen, C₁-C₆ alkyl, hydroxy, amino, nitro, trifluoromethyl, cyano, COOC₁₋₄ alkyl, CO₁₋₄ alkyl, CO₁₋₄ aryl, SO₂(C₁₋₄ alkyl), SO₂ arylamino and/or C₁-C₆ alkoxy, and

p is 0, 1 or 2,

as well as isomers and salts thereof.

Also quite especially effective are those compounds of general formula I, in which

R¹ stands for phenyl or pyridyl that is optionally substituted by the same or a different component in one or more places with halogen, hydroxy, C₁-C₆ alkyl, amino, nitro, trifluoromethyl, —O—Si(C₁-C₆ alkyl)₃, cyano, COOC alkyl, CO₁₋₄ alkyl, CO₁₋₄ aryl, SO₂(C₁₋₄ alkyl), SO₂ aryl and/or C₁-C₆ alkoxy, and

R² stands for hydrogen, hydroxy, C₁₋₆ alkyloxy, aryloxy, trifluoromethyl, nitro, halogen, cyano or the group —SOₚR⁶, —SO₂NR⁷R⁸, or —COR⁹, or

R¹ stands for hydrogen, hydroxy, C₁₋₆ alkyloxy, aryloxy, trifluoromethyl, nitro, halogen, cyano or the group —SOₚR⁶, —SO₂NR⁷R⁸, or —COR⁹, and

R² stands for phenyl or pyridyl that is optionally substituted in one or more places with halogen, hydroxy, C₁-C₆ alkyl, amino, nitro, trifluoromethyl, cyano, COOC₁₋₄ alkyl, CO₁₋₄ alkyl, CO₁₋₄ aryl, SO₂(C₁₋₄ alkyl), SO₂ aryl and/or C₁-C₆ alkoxy,

R⁴ and R⁵ stand for hydrogen, hydroxy, C₁₋₆ alkoxy, aryloxy, —CF₃, nitro, halogen, cyano or the group —SOₚR⁶, —SO₂NR⁷R⁸ or —COR⁹; or for aryl or heteroaryl that is optionally substituted in one or more places with halogen, hydroxy, C₁-C₆ alkyl, amino, nitro, trifluoromethyl, cyano, COOC₁₋₄ alkyl, CO₁₋₄ alkyl, CO₁₋₄ aryl, SO₂(C₁₋₄ alkyl), SO₂ aryl and/or C₁-C₆ alkoxy,

R³ stands for oxygen or the group —NOR⁹, or

R¹ and R² or

R⁴ and R⁵ optionally form another C₃-C₆-membered ring,

R⁶ stands for hydrogen or C₁₋₁₈ alkyl,

R⁷ and R⁸ are the same or different and stand for hydrogen or for C₁₋₁₈ alkyl, aryl, heteroaryl or acyl that is optionally substituted in one or more places with hydroxy, halogen and/or amino; or C₁₋₁₈ alkyl, C₃₋₈ cycloalkyl or C₃₋₈ cycloalkenyl that is optionally interrupted by one or more oxygen atoms, or

R⁷ or R8 together with the nitrogen atom of the amino group forms a C₃₋₈ cycloalkyl, which can contain one or more other heteroatoms,

R⁹ stands for hydrogen or for C₁₋₆ alkyl that is optionally substituted in one or more places with hydroxy, halogen, C₁₋₄ alkoxy, amino, nitro, trifluoromethyl, cyano, COOC₁₋₄ alkyl, CO₁₋₄ alkyl, CO₁₋₄ aryl, SO₂(C₁₋₄ alkyl) and/or SO₂-aryl; or for aryl or heteroaryl that is optionally substituted in one or more places with halogen, C₁-C₆ alkyl, hydroxy, amino, nitro, trifluoromethyl, cyano, COOC₁₋₄ alkyl, CO₁₋₄ alkyl, CO₁₋₄ aryl, SO₂(C₁₋₄ alkyl), SO₂ arylamino and/or C₁-C₆ alkoxy, and

p is 0, 1 or 2,

as well as isomers and salts thereof.

Also preferred, since they are highly effective, are those compounds of general formula I, in which

R¹ stands for phenyl or pyridyl that is optionally substituted by the same or a different component in one or more places with halogen, hydroxy, C₁-C₆ alkyl,

amino, nitro, trifluoromethyl, —O—Si(C₁-C₆ alkyl)₃, cyano, COOC₁₋₄ alkyl, CO ₄ alkyl, CO₁₋₄ aryl, SO₂(CO₁₋₄ alkyl), SO₂ aryl and/or C₁-C₆ alkoxy,

R² stands for hydrogen,

R⁴ and R⁵ stand for hydrogen, hydroxy, C₁₋₆ alkoxy, aryloxy, —CF₃, nitro, halogen, cyano, or the group —SOₚR⁶, —SO₂NR⁷R⁸, or —COR⁹; or for aryl or heteroaryl that are optionally substituted in one or more places with halogen, hydroxy, C₁-C₆ alkyl, C₁₋₆ alkoxy, amino, nitro, trifluoromethyl, cyano, COOC₁₋₄ alkyl, CO₁₋₄ alkyl, CO₁₋₄ aryl, SO₂(C₁₋₄ alkyl), SO₂ aryl and/or C₁-C₆ alkoxy,

R³ stands for oxygen or the group —NOR⁹, or

R⁴ and R⁵ optionally form another C₃-C₆-membered ring,

R⁶ stands for hydrogen or C₁₋₁₈ alkyl,

R⁷ and R8 are the same or different and stand for hydrogen or for C₁₋₁₈ alkyl, aryl, heteroaryl or acyl that is optionally substituted in one or more places with hydroxy, halogen and/or amino; or C₁₋₁₈ alkyl, C₃₋₈ cycloalkyl or C₃₋₈ cycloalkenyl that is optionally interrupted by one or more oxygen atoms, or

R⁷ or R⁸ together with the nitrogen atom of the amino group forms a C₃₋₈ cycloalkyl, which can contain one or more other heteroatoms,

R⁹ stands for hydrogen or for C₁₋₆ alkyl that is optionally substituted in one or more places with hydroxy, halogen, C₁₋₄ alkoxy, amino, nitro, trifluoromethyl, cyano, COOC₁₋₄ alkyl, CO₁₋₄ alkyl, CO₁₋₄ aryl, SO₂(C₁₋₄ alkyl) and/or SO₂-aryl; or for aryl or heteroaryl that is substituted in one or more places with halogen, C₁-C₆ alkyl, hydroxy, amino, nitro, trifluoromethyl, cyano, COOC₁₋₄ alkyl, CO₁₋₄ alkyl, CO₁₋₄ aryl, SO₂(C₁₋₄ alkyl), SO₂ arylamino and/or C₁-C₆ alkoxy, and

p is 0, 1 or 2,

as well as isomers and salts thereof.

The compounds of general formula I, in which

R¹ stands for phenyl that is optionally substituted by the same or a different component in one or more places with halogen, hydroxy, C₁-C₆ alkyl, amino, nitro, trifluoromethyl, —O—Si(C₁-C₆ alkyl)₃, cyano, COOC₁₋₄ alkyl, CO₁₋₄ alkyl, CO₁₋₄ aryl, SO₂(C₁₋₄ alkyl), SO₂ aryl and/or C₁-C₆ alkoxy,

R² stands for hydrogen,

R⁴ and R⁵ stand for hydrogen, hydroxy, C₁₋₆ alkoxy, aryloxy, trifluoromethyl, nitro, halogen, cyano or the group —SOₚR⁶, —SO₂NR⁷R⁸, or —COR⁹; or for aryl or heteroaryl that is optionally substituted in one or more places with halogen, hydroxy, C₁-C₆ alkyl, C₁₋₆ alkoxy, amino, nitro, trifluoromethyl, cyano, COOC₁₋₄ alkyl, CO₁₋₄ alkyl, CO₁₋₄ aryl, SO₂(C₁₋₄ alkyl), SO₂ aryl and/or C₁-C₆ alkoxy,

R³ stands for oxygen or the group —NOR⁹, or

R⁴ and R⁵ optionally form another C₃-C₆-membered ring,

R⁶ stands for hydrogen or C₁₋₁₈ alkyl,

R⁷ and R8 are the same or different and stand for hydrogen or for C₁₋₁₈ alkyl, aryl, heteroaryl or acyl that is optionally substituted in one or more places with hydroxy, halogen and/or amino; or C₁₋₁₈ alkyl, C₃₋₈ cycloalkyl or C₃₋₈ cycloalkenyl that is optionally interrupted by one or more oxygen atoms, or

R⁷ or R⁸ together with the nitrogen atom of the amino group forms a C₃₋₈ cycloalkyl, which can contain one or more other heteroatoms,

R⁹ stands for hydrogen or for C₁₋₆ alkyl that is optionally substituted in one or more places with hydroxy, halogen, C₁₋₄ alkoxy, amino, nitro, trifluoromethyl, cyano, COOC₁₋₄ alkyl, CO₁₋₄ alkyl, CO₁₋₄ aryl, SO₂(C₁₋₄ alkyl) and/or So₂ aryl; or for aryl or heteroaryl that is optionally substituted in one or more places with halogen, C₁-C₆ alkyl, hydroxy, amino, nitro, trifluoromethyl, cyano, COOC₁₋₄ alkyl, CO₁₋₄ alkyl, CO₁₋₄ aryl, SO₂(C₁₋₄ alkyl), SO₂-arylamino and/or C₁-C₆ alkoxy, and

p is 0, 1 or 2,

as well as isomers and salts thereof,

also-exhibit surprisingly good action.

Those compounds of general formula I, in which

R¹ stands for phenyl that is optionally substituted by the same or a different component in one or more places with halogen, hydroxy, C₁-C₆ alkyl, amino, nitro, trifluoromethyl, —O—Si(C₁-C₆ alkyl)₃, cyano, COOC₁₋₄ alkyl, C₁₋₄ alkyl, CO₁₋₄ aryl, SO₂(C₁₋₄ alkyl), SO₂ aryl and/or C₁-C₆ alkoxy,

R² stands for hydrogen,

R⁴ and R⁵ stand for hydrogen,

R³ stands for oxygen,

as well as isomers and salts thereof,

exhibit excellent properties.

Those compounds of general formula I, in which

R¹ stands for phenyl that is optionally substituted by the same or a different component in one or more places with halogen, hydroxy, C₁₋₄ alkyl, amino, nitro, trifluoromethyl, O—Si(C₁-C₆ alkyl)₃, cyano or C₁-C₄ alkoxy,

R² stands for hydrogen,

R⁴ and R⁵ stand for hydrogen,

R³ stands for oxygen,

as well as isomers and salts thereof,

also exhibit quite excellent properties.

The compounds according to the invention essentially inhibit cyclin-dependent kinases, upon which their action is based, for example, against cancer, such as solid tumors and leukemia; auto-immune diseases, such as psoriasis, alopecia and multiple sclerosis; chemotherapy agent-induced alopecia and mucositis; cardiovascular diseases, such as stenoses, arterioscleroses and restenoses; infectious diseases, such as, e.g., those caused by unicellular parasites, such as trypanosoma, toxoplasma or plasmodium, or those caused by fungi; nephrological diseases, such as, e.g., glomerulonephritis; chronic neurodegenerative diseases, such as Huntington's disease, amyotrophic lateral sclerosis, Parkinson's disease, AIDS dementia and Alzheimer's disease; acute neurodegenerative diseases, such as ischemias of the brain and neurotraumas; viral infections, such as, e.g., cytomegalic infections, herpes, hepatitis B and C, and HIV diseases.

The eukaryotic cell division cycle ensures the duplication of the genome and its distribution to the daughter cells by passing through a coordinated and regulated sequence of events. The cell cycle is divided into four successive phases: The G1 phase represents the time before the DNA replication, in which the cell grows and is sensitive to external stimuli. In the S phase, the cell replicates its DNA, and in the G2 phase, preparations are made for entry into mitosis. In mitosis (M phase), the replicated DNA separates, and cell division is complete.

The cyclin-dependent kinases (CDKs), a family of Ser/Thr kinases, whose members require the binding of a cyclin (Cyc) as a regulatory subunit in order for them to activate, drive the cell through the cell cycle. Different CDK/Cyc pairs are active in the various phases of the cell cycle. CDK/Cyc pairs that are important to the basic function of the cell cycle are, for example, CDK4(6)/CycD, CDK2/CycE, CDK2/CycA, CDK1/CycA and CDK1/CycB. Some members of the CDK-enzyme family have a regulatory function by influencing the activity of the above-mentioned cell cycle CDKs, while no specific function could be associated with other members of the CDK enzyme family. One of the latter, CDK5, is distinguished in that it has an atypical regulatory subunit (p35) that-deviates from the cyclins, and its activity is highest in the brain.

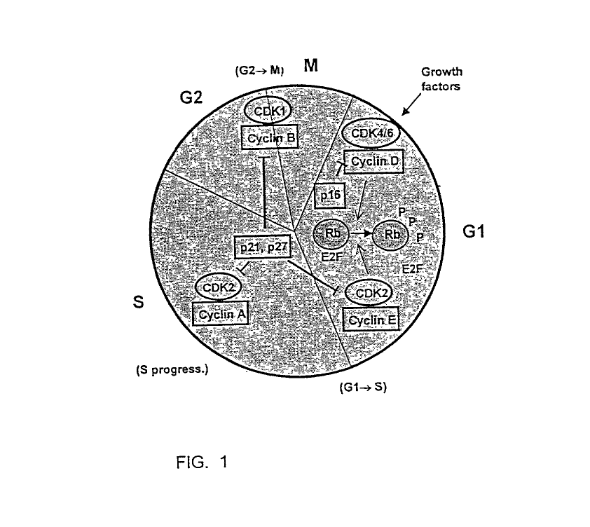

The entry into the cell cycle and the passage through the “restriction points,” which marks the independence of a cell from further growth signals for the completion of the cell division that has begun, are controlled by the activity of the CDK4(6)/CycD and CDK2/CycE complexes. The essential substrate of these CDK complexes is the retinoblastoma protein (Rb), the product of the retinoblastoma tumor suppressor gene. Rb is a transcriptional co-repressor protein. In addition to other, still largely little understood mechanisms, Rb binds and inactivates transcription factors of the E2F type and forms transcriptional repressor complexes with histone-deacetylases (HDAC) (Zhang, H. S. et al. (2000). Exit from G1 and S Phase of the Cell Cycle is Regulated by Repressor Complexes Containing HDAC-Rb-hSWI/SNF and Rb-hSWI/SNF. Cell 101, 79-89). By the phosphorylation of Rb by CDKs, bonded E2F transcription factors are released and result in transcriptional activation of genes, whose products are required for the DNA synthesis and the progression through the S-phase. In addition, the Rb-phosphorylation brings about the breakdown of the Rb-HDAC complexes, by which additional genes are activated. The phosphorylation of Rb by CDK's is to be treated as equivalent to exceeding the “restriction points.” For the progression through the S-phase and its completion, the activity of the CDK2/CycE and CDK2/CycA complexes is necessary, e.g., the activity of the transcription factors of the E2F type is turned off by means of phosphorylation by CDK2/CycA as soon as the cells are entered into the S-phase. After replication of DNA is complete, the CDK1 in the complex with CycA or CycB controls the entry into and the passage through phases G2 and M (FIG. 1).

According to the extraordinary importance of the cell-division cycle, the passage through the cycle is strictly regulated and controlled. The enzymes that are necessary for the progression through the cycle must be activated at the correct time and are also turned off again as soon as the corresponding phase is passed. Corresponding control points (“checkpoints”) stop the progression through the cell cycle if DNA damage is detected, or the DNA replication or the creation of the spindle device is not yet completed.

The activity of the CDKs is controlled directly by various mechanisms, such as synthesis and degradation of cyclins, complexing of the CDKs with the corresponding cyclins, phosphorylation and dephosphorylation of regulatory Thr and Tyr radicals, and the binding of natural inhibitory proteins. While the amount of protein of the CDKs in a proliferating cell is relatively constant, the amount of the individual cyclins oscillates with the passage through the cycle. Thus, for example, the expression of CycD during the early G1 phase is stimulated by growth factors, and the expression of CycE is induced after the “restriction points” are exceeded by the activation of the transcription factors of the E2F type. The cyclins themselves are degraded by the ubiquitin-mediated proteolysis. Activating and inactivating phosphorylations regulate the activities of the CDKs, for example phosphorylate CDK-activating kinases (CAKs) Thr160/161 of the CDK1, while, by contrast, the families of Wee1/Myt1 inactivate kinases CDK1 by phosphorylation of Thr14 and Tyr15. These inactivating phosphorylations can be destroyed in turn by cdc25 phosphatases. The regulation of the activity of the CDK/Cyc complexes by two families of natural CDK inhibitor proteins (CKIs), the protein products of the p21 gene family (p21, p27, p57) and the p16 gene family (p15, p16, p18, p19) is very significant. Members of the p21 family bind to cyclin complexes of CDKs 1,2,4,6, but inhibit only the complexes that contain CDK1 or CDK2. Members of the p16 family are specific inhibitors of the CDK4- and CDK6 complexes.

The plane of control point regulation lies above this complex direct regulation of the activity of the CDKs. Control points allow the cell to track the orderly sequence of the individual phases during the cell cycle. The most important control points lie at the transition from G1 to S and from G2 to M. The G1 control point ensures that the cell does not initiate any DNA synthesis unless it has proper nutrition, interacts correctly with other cells or the substrate, and its DNA is intact. The G2/M control point ensures the complete replication of DNA and the creation of the mitotic spindle before the cell enters into mitosis. The G1 control point is activated by the gene product of the p53 tumor suppressor gene. p53 is activated after detection of changes in metabolism or the genomic integrity of the cell and can trigger either a stopping of the cell cycle progression or apoptosis. In this case, the transcriptional activation of the expression of the CDK inhibitor protein p21 by p53 plays a decisive role. A second branch of the G1 control point comprises the activation of the ATM and Chk1 kinases after DNA damage by UV light or ionizing radiation and finally the phosphorylation and the subsequent proteolytic degradation of the cdc25A phosphatase (Mailand, N. et al. (2000). Rapid Destruction of Human cdc25A in Response to DNA Damage. Science 288, 1425-1429). A shutdown of the cell cycle results from this, since the inhibitory phosphorylation of the CDKs is not removed. After the G2/M control point is activated by damage of the DNA, both mechanisms are involved in a similar way in stopping the progression through the cell cycle.

The loss of the regulation of the cell cycle and the loss of function of the control points are characteristics of tumor cells. The CDK-Rb signal path is affected by mutations in over 90% of human tumor cells. These mutations, which finally result in inactivating phosphorylation of the RB, include the over-expression of D- and E-cyclins by gene amplification or chromosomal translocations, inactivating mutations or deletions of CDK inhibitors of the p16 type, as well as increased (p27) or reduced (CycD) protein degradation. The second group of genes, which are affected by mutations in tumor cells, codes for components of the control points. Thus p53, which is essential for the G1 and G2/M control points, is the most frequently mutated gene in human tumors (about 50%). In tumor cells that express p53 without mutation, it is often inactivated because of a greatly increased protein degradation. In a similar way, the genes of other proteins that are necessary for the function of the control points are affected by mutations, for example ATM (inactivating mutations) or cdc25 phosphatases (over-expression).

Convincing experimental data indicate that CDK2/cyc complexes occupy a decisive position during the cell cycle progression: (1) Both dominant-negative forms of CDK2, such as the transcriptional repression of the CDK2 expression by anti-sense oligonucleotides, produce a stopping of the cell cycle progression. (2) The inactivation of the CycA gene in mice is lethal. (3) The disruption of the function of the CDK2/CycA complex in cells by means of cell-permeable peptides resulted in tumor cell-selective apoptosis (Chen, Y. N. P. et al. (1999). Selective Killing of Transformed Cells by Cyclin/Cyclin-Dependent Kinase 2 Antagonists. Proc. Natl. Acad. Sci. USA 96, 4325-4329).

Changes of the cell cycle control play a role not only in carcinoses. The cell cycle is activated by a number of viruses, both by transforming viruses as well as by non-transforming viruses, to make possible the reproduction of viruses in the host cell. The false entry into the cell cycle of normally post-mitotic cells is associated with various neurodegenerative diseases. The mechanisms of the cell cycle regulation, their changes in diseases and a number of approaches to develop inhibitors of the cell cycle progression and especially the CDKs were already described in a detailed summary in several publications (Sielecki, T. M. et al. (2000). Cyclin-Dependent Kinase Inhibitors: Useful Targets in Cell Cycle Regulation. J. Med. Chem. 43, 1-18; Fry, D. W. & Garrett, M. D. (2000). Inhibitors of Cyclin-Dependent Kinases as Therapeutic Agents for the Treatment of Cancer. Curr. Opin. Oncol. Endo. Metab. Invest. Drugs 2, 40-59; Rosiania, G. R. & Chang, Y. T. (2000). Targeting Hyperproliferative Disorders with Cyclin-Dependent Kinase Inhibitors. Exp. Opin. Ther. Patents 10, 215-230; Meijer L. et al. (1999). Properties and Potential Applications of Chemical Inhibitors of Cyclin-Dependent Kinases. Pharmacol. Ther. 82, 279-284; Senderowicz, A. M. & Sausville E. A. (2000). Preclinical and Clinical Development of Cyclin-Dependent Kinase Modulators. J. Natl. Cancer Inst. 92, 376-387).

To use the compounds according to the invention as pharmaceutical agents, the latter are brought into the form of a pharmaceutical preparation, which in addition to the active ingredient contains pharmaceutical, organic or inorganic inert support media, such as, for example, water, gelatin, gum arabic, lactose, starch, magnesium stearate, talc, vegetable oils, polyalkylene glycols, etc., that are suitable for enteral or parenteral administration. The pharmaceutical preparations can be present in solid form, for example as tablets, coated tablets, suppositories, capsules, or in liquid form, for example as solutions, suspensions, or emulsions. Moreover, they optionally contain adjuvants, such as preservatives, stabilizers, wetting agents or emulsifiers; salts for changing the osmotic pressure or buffers.

These pharmaceutical preparations are also subjects of this invention.

For parenteral administration, especially injection solutions or suspensions, especially aqueous solution of active compounds in polyhydroxyethoxylated castor oil are suitable.

As carrier systems, surface-active adjuvants such as salts of bile acids or animal or plant phospholipids, but also mixtures thereof as well as liposomes or their components, can also be used.

For oral administration, especially tablets, coated tablets or capsules with talc and/or hydrocarbon vehicles or binders, such as, for example, lactose, corn or potato starch, are suitable. The administration can also be carried out in liquid form, such as, for example, as a juice, to which optionally a sweetener is added.

Enteral, parenteral and oral administrations are also subjects of this invention.

The dosage of the active ingredients can vary depending on the method of administration, age and weight of the patient, type and severity of the disease to be treated and similar factors. The daily dose is 0.5-1000 mg, preferably 50-200 mg, whereby the dose can be given as a single dose to be administered once or divided into two or more daily doses.

Subjects of this invention are also the use of compounds of general formula I for the production of a pharmaceutical agent for treating cancer, auto-immune diseases, cardiovascular diseases, chemotherapy agent-induced alopecia and mucositis, infectious diseases, nephrological diseases, chronic and acute neurodegenerative diseases and viral infections, whereby cancer is defined as solid tumors and leukemia; auto-immune diseases are defined as psoriasis, alopecia and multiple sclerosis; cardiovascular diseases are defined as stenoses, arterioscleroses and restenoses; infectious diseases are defined as diseases that are caused by unicellular parasites; nephrological diseases are defined as glomerulonephritis; chronic neurodegenerative diseases are defined as Huntington's disease, amyotrophic lateral sclerosis, Parkinson's disease, AIDS dementia and Alzheimer's disease; acute neurodegenerative diseases are defined as ischemias of the brain and neurotraumas; and viral infections are defined as cytomegalic infections, herpes, hepatitis B or C, and HIV diseases.

Subjects of this invention are also pharmaceutical agents for treating the above-cited diseases, which include at least one compound according to general formula I, as well as pharmaceutical agents with suitable formulation substances and vehicles.

The compounds of general formula I according to the invention are, i.a., excellent inhibitors of the cyclin-dependent kinases, such as CDK1, CDK2, CDK3, CDK4, CDK5, CDK6, CDK7, CDK8 and CDK9, as well as the glycogen-synthase-kinase (GSK-3β).

If the production of the starting compounds is not described, these compounds are known or can be produced analogously to known compounds or to processes that are described here. It is also possible to perform all reactions that are described here in parallel reactors or by means of combinatory operating procedures.

The isomer mixtures can be separated into the enantiomers or E/Z isomers according to commonly used methods, such as, for example, crystallization, chromatography or salt formation.

The production of the salts is carried out in the usual way by a solution of the compound of formula I being mixed with the equivalent amount of or excess base or acid, which optionally is in solution, and the precipitate being separated or the solution being worked up in the usual way.

The following examples explain the production of the compounds according to the invention.

The production of the compounds according to the invention is carried out essentially according to the following diagram:

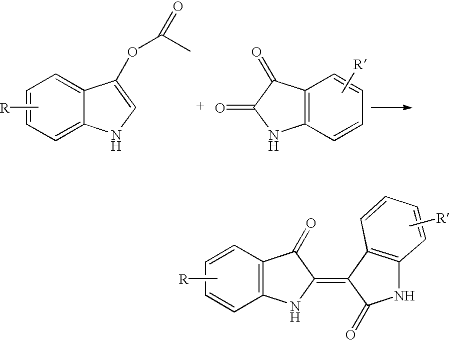

Hereinafter, R stands for R⁴ and R⁵, and R′ stands for R¹ and R² of general formula I.

The indirubins can also be further functionalized by the incorporation of additional substituents. As an alternative to this, first the precursor compounds (indoxyl acetates, isatins) can also be derivatized. The creation of indirubins is then carried out by condensation:

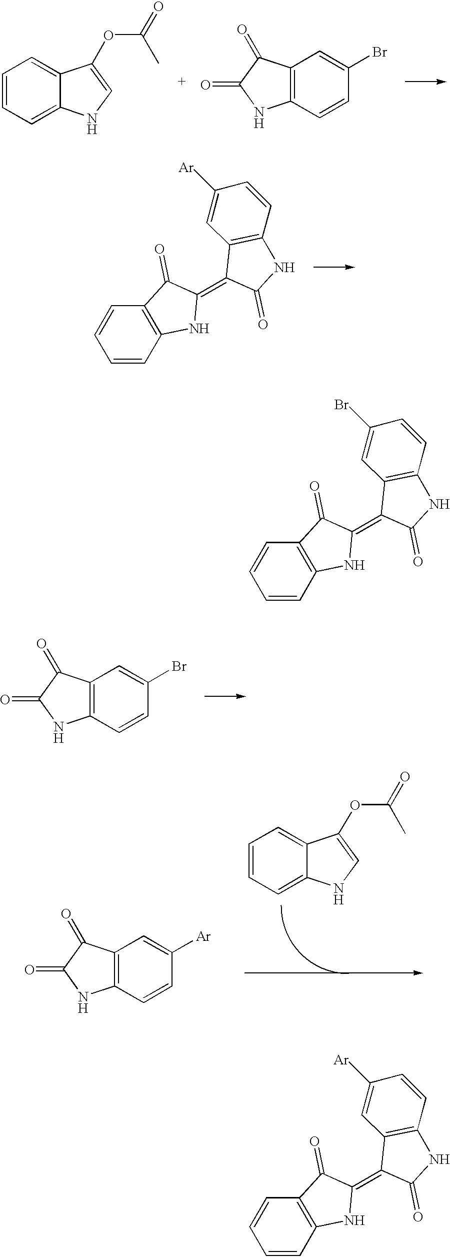

Moreover, the introduction of substituents can also be carried out in a transformation that is downstream to the formation of indirubins.

The syntheses of compounds can be performed either as individual reactions or as parallel reactions or in a combinatory synthesis scheme.

The isomer mixtures can be separated into enantiomers or E/Z isomers according to commonly used methods, such as, for example, crystallization, chromatography or salt formation.

The production of salts is carried out in the usual way by a solution of the compound of formula I being mixed with the equivalent amount of or excess base or acid, which optionally is in solution, and the precipitate being separated or the solution being worked up in the usual way.

If the production of the starting compounds is not described, the latter are known or can be produced analogously to known compounds or processes that are described here.

The synthesis of indirubins can be carried out, for example, analogously to the reaction that is described in the literature by reaction of the corresponding isatin with an indoxyl acetate. To increase the yield and to minimize secondary reactions, the reaction should be carried out under a cover gas (e.g., argon, nitrogen). The general synthesis technique is described in the literature (G. A. Russeil, G. Kaupp, J. Am. Chem. Soc. 1969, 91, 3851-3859).

The synthesis of indoxyl acetates is carried out analogously to the process that is mentioned in the literature (Friedlaender, Bruckner, Justus Liebigs Ann. Chem. 1912, 388). If the compounds were commercially available, the latter were used for synthesis without previous purification.

The palladium-catalyzed coupling is an established method for linking aromatic or other kinds of sp² centers with, for example, aryl radicals. The basic principle of this reaction is known in the literature and is the subject of extensive reviews (cf.); depending on the catalyst that is used (for example palladium or nickel) or the leaving group of the radical that is to be introduced (for example magnesium, boron, tin or zinc compounds), widely varying name-reactions are distinguished.

The introduction of other substituents into the indirubin building blocks via such a palladium-catalyzed coupling is novel and not known in the literature. It has turned out in the reactions, surprisingly enough, that in the case of the Suzuki couplings, the most promising yields were obtained by the use of palladium(II) acetate in combination with tri-ortho-tolylphosphine.

The synthesis of oximes (see diagram) is carried out by reaction of the corresponding carbonyl compound with hydroxylamine, analogously to the instructions that are known in the literature (C. Li et al., Bull. Chem. Soc. Jpn. (1996), 69, 1621-1627).

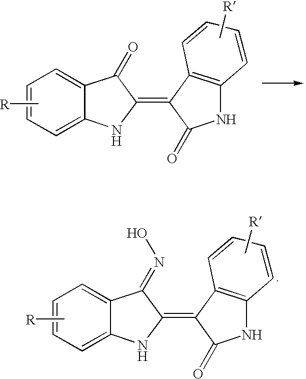

The production of oxime-ethers (see diagram) is carried out from the corresponding oximes by base-catalyzed etherification in the presence of an alkylating agent, analogously to the instructions that are known in the literature (cf. DE 283726, C. Li, Y. Go et al. Bull Chem. Soc. Jpn. 1996, 69, 1621-1628). As a base for this reaction, inorganic or organic bases can be used. As a solvent, protic or aprotic polar media are used.

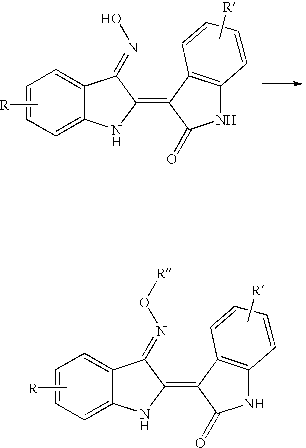

In the diagram, R has the meaning of R⁴ and R⁵, R″ has the meaning of R⁹, and R′ has the meaning of R¹ and R² of general formula I.

The following examples explain the production of starting compounds without limiting the scope of the claimed compounds to these examples.

### EXAMPLE 1.0

5-(4-Methoxyphenyl)-indirubin

342 mg (1.0 mmol) of 5-bromoindirubin is introduced under nitrogen into 5 ml of toluene/ethanol (4:1) and 2 ml of dimethyl sulfoxide. 127 mg (3 mmol) of lithium chloride, 304 mg (2 mmol) of 4-methoxyphenylboronic acid, 1.3 ml (2.6 mmol) of 2N sodium carbonate solution in water are added in succession. Finally, 15.2 mg (0.05 mmol) of tri-o-tolyl-phosphine and 11.2 mg (0.05 mmol) of palladium(II) acetate are added to it, and the reaction mixture is heated to 90° C. After 24 hours of stirring, 5 mol % of tri-o-tolylphosphine and palladium(II) acetate are added again. After 48 hours, the reaction is worked up. For working-up, water and ethyl acetate are added to the reaction mixture, and the phases are separated. The aqueous phase is extracted twice more with 3 ml of ethyl acetate each, the combined organic phases are dried on sodium sulfate, and the solvent is removed in a vacuum. After chromatographic purification on silica gel, the desired compound is isolated in the form of deep-violet crystals (254 mg, 69%).

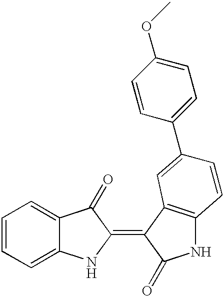

¹H NMR (DMSO-D₆): 67 3.83 (s, 3H), 6.97 (d, 1H, J =8 Hz), 7.03-7.10 (m, 3H), 7.45 (d, 1H, J =7 Hz), 7.53 (dd, 1H), 7.60 1H), 7.62 (d, 2H), 7.70 (d, 1H), 9.12 (d, 1H), 10.95 (s, 1H), 11.06 (s, 1H).

MS (Cl, NH₃) %: 369 (100) (M+H), 263 (20), 240 (7).

Melting point: >300° C. (ethyl acetate)

The following compounds are also produced by a similar method:

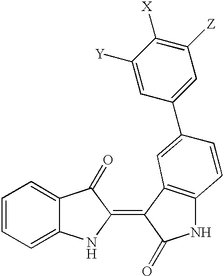

| Example | | | | Melting Point [° C.] |
|---|---|---|---|---|
| No. | X | Y | Z | and MS/NMR |
| 1.1 | H | H | H | MS: 338 (M+, 100); 310 |
| | | | | (47) |
| 1.2 | H | —O—Si(CH₃)₂— | H | 1H NMR (DMSO-D6): δ |
| | | C(CH₃)₃ | | 0.38 (s, 6H, Si(CH₃)₂, |
| | | | | 102 (9H, (CH₃)6.86(dd, |
| | | | | 1H), 6.97-7.10 (m, 2H, |
| | | | | 7.12(s, 1H), 7.29 (d, |
| | | | | 1H), 7.38-7.48 (m, |
| | | | | 2H), 7.53-7.64 (m, |
| | | | | 2H), 7.68 (d, 1H), |
| | | | | 9.14 (d, 1H), 10.94 |
| | | | | (s, 1H), 11.05 (s, 1H). |
| 1.3 | H | —CF₃ | —CF₃ | 1H-NMR(DMSO-D6): 7.08 |
| | | | | (m, 2H), 7.45 (d, 1H), |
| | | | | 7.61 (m, 1H), 7.70- |
| | | | | 7.81 (m, 2H), 8.08 (s, |
| | | | | H), 8.28 (s, 2H), 9.22 |
| | | | | (s, 1H), 11.06 (s, |
| | | | | 1H), 11.08 (s, 1H). |
| 1.4 | Cl | H | H | 1H-NMR(DMSO-D6): 7.03 |
| | | | | (m, 2H), 7.44 (m, 1H), |
| | | | | 7.53-7.53 (m, 8H), |
| | | | | 9.15 (s, 1H), 11.00 |
| | | | | (s, 1H), 11.08 (s, 1H). |
| 1.5 | —C(CH₃)₃ | H | H | 395(11) [M+H]; |
| | | | | 263(100), 135(5) |

Description of the Figure

FIG. 1 shows the simplified diagram of cell cycle regulation in vertebrates.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

In the foregoing and in the following examples, all temperatures are set forth uncorrected in degrees Celsius and, all parts and percentages are by weight, unless otherwise indicated.

The entire disclosure of all applications, patents and publications, cited above, and of corresponding German application No. 100 61 162.1, filed Nov. 30, 2000 is hereby incorporated by reference.

The following examples describe the biological action of the compounds according to the invention without limiting the invention to these examples.

### EXAMPLE 1

CDK2/CycE Kinase Assay

Recombinant CDK2- and CycE-GST-fusion proteins, purified from baculovirus-infected insect cells (Sf9), were obtained by Dr. Dieter Marmé, Klinik für Tumorbiologie [Clinic for Tumor Biology], Freiburg. Histone IIIS, which was used as a kinase substrate, was purchased by the Sigma Company.

CDK2/CycE (50 ng/measuring point) was incubated for 15 minutes at 22° C. in the presence of various concentrations of test substances (0 μm, as well as within the range of 0.01-100 μm) in assay buffer [50 mmol of tris/HCl pH 8.0, 10 mmol of MgCl₂, 0.1 mmol of Na ortho-vanadate, 1.0 mmol of dithiothreitol, 0.5 μm of adenosine triphosphate (ATP), 10 μg/measuring point of histone IIIS, 0.2 μCi/measuring point of ³³P-gamma ATP, 0.05% NP40, 12.5% dimethyl sulfoxide]. The reaction was stopped by adding EDTA solution (250 mmol, pH 8.0, 14 μl/measuring point).

From each reaction batch, 10 μl was applied to P30 filter strips (Wallac Company), and non-incorporated ³³P-ATP was removed by subjecting the filter strips to three washing cycles for 10 minutes each in 0.5% phosphoric acid. After the filter strips were dried for one hour at 70° C., the filter strips were covered with scintillator strips (MeltiLex™ A, Wallac Company) and baked for one hour at 90° C. The amount of incorporated ³³P (substrate phosphorylation) was determined by scintillation measurement in a gamma-radiation measuring device (Wallac).

### EXAMPLE 2

CDK1/CycB Kinase Assay

Recombinant CDK1- and CycB-GST fusion proteins, purified from baculovirus-infected insect cells (Sf9), were obtained by Dr. Dieter Marmé, Klinik für Tumorbiologie Freiburg. Histone IIIS, which was used as a kinase substrate, was purchased by the Sigma Company.

CDK1/CycB (50 ng/measuring point) was incubated for 15 minutes at 22° C. in the presence of various concentrations of test substances (0 μm, as well as within the range of 0.01-100 μm) in assay buffer [50 mmol of tris/HCl pH 8.0, 10 mmol of MgCl₂, 0.1 mmol of Na ortho-vanadate, 1.0 mmol of dithiothreitol, 0.5 μm of adenosine triphosphate (ATP), 10 μg/measuring point of histone IIIS, 0.2 μCi/measuring point of ³³P-gamma ATP, 0.05% NP40, 12.5% dimethyl sulfoxide]. The reaction was stopped by adding EDTA solution (250 mmol, pH 8.0, 14 μl/measuring point).

From each reaction batch, 10 μl was applied to P30 filter strips (Wallac Company), and non-incorporated ³³P-ATP was removed by subjecting the filter strips to three washing cycles for 10 minutes each in 0.5% phosphoric acid. After the filter strips were dried for one hour at 70° C., the filter strips were covered with scintillator strips (MeltiLex™ A, Wallac Company) and baked for one hour at 90° C. The amount of incorporated ³³P (substrate phosphorylation) was determined by scintillation measurement in a gamma-radiation measuring device (Wallac).

### EXAMPLE 3

CDK4/CycD1 Kinase Assay

Recombinant CDK4- and CycD1-GST fusion proteins, purified from baculovirus-infected insect cells (Sf9), were obtained by Dr. Dieter Marmé, Klinik für Tumorbiologie Freiburg. The kinase substrate, a GST-fusion protein of the 20 kD C-terminal fragment of the Rb protein, was obtained by Dr. Dieter Marmé, Klinik für Tumorbiologie Freiburg.

CDK4/CycD1 (200 ng/measuring point) was incubated for 15 minutes at 22° C. in the presence of various concentrations of test substances (0 μm, as well as within the range of 0.01-100 μm) in assay buffer [50 mmol of tris/HCl pH 8.0, 10 mmol of MgCl₂, 0.1 mmol of Na ortho-vanadate, 1.0 mmol of dithiothreitol, 0.5 μm of adenosine triphosphate (ATP), 1 μg/measuring point of C-terminal Rb-GST-fusion protein, 0.2 μCi/measuring point of ³³P-gamma ATP, 0.05% NP40, 12.5% dimethyl sulfoxide]. The reaction was stopped by adding EDTA solution (250 mmol, pH 8.0, 14 μl/measuring point).

From each reaction batch, 10 μl was applied to P30 filter strips (Wallac Company), and non-incorporated ³³P-ATP was removed by subjecting the filter strips to three washing cycles for 10 minutes each in 0.5% phosphoric acid. After the filter strips were dried for one hour at 70° C., the filter strips were covered with scintillator strips (MeltiLex™ A, Wallac Company) and baked for one hour at 90° C. The amount of incorporated ³³P (substrate phosphorylation) was determined by scintillation measurement in a gamma-radiation measuring device (Wallac).

### EXAMPLE 4

Proliferation Assay

Cultivated human tumor cells (as indicated) were flattened out at a density of 5000 cells/measuring point in a 96-well multititer plate in 200 μl of the corresponding growth medium. After 24 hours, the cells of one plate (zero-point plate) were colored with crystal violet (see below), while the medium of the other plates was replaced by fresh culture medium (200 μl), to which the test substances were added in various concentrations (0 μm, as well as in the range of 0.01-30 μm; the final concentration of the solvent dimethyl sulfoxide was 0.5%). The cells were incubated for 4 days-in the presence of test substances. The cell proliferation was determined by coloring the cells with crystal violet: the cells were fixed by adding 20 μl/measuring point of a 11% glutaric aldehyde solution for 15 minutes at room temperature. After subjecting the fixed cells to three washing cycles with water, the plates were dried at room temperature. The cells were colored by adding 100 μL/measuring point of a 0.1% crystal violet solution (pH was set at 3 by adding acetic acid). After subjecting the colored cells to three washing cycles with water, the plates were dried at room temperature. The dye was dissolved by adding 100 μL/measuring point of a 10% acetic acid solution. The extinction was determined by photometry at a wavelength of 595 nm. The change of cell growth, in percent, was calculated by standardization of the measured values to the extinction values of the zero-point plate (=0%) and the extinction of the untreated (0 μm) cells (=100%).

Proof of Superiority of the Compounds According to the Invention Compared to the Known Compounds

To prove the superiority of the compounds according to the invention compared to the known compounds, the compounds according to the invention were compared to structurally-similar known compounds both in the enzyme test and in the cell test. The result is cited in the following table:

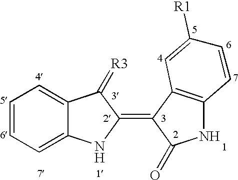

The preceding examples can be repeated with similar success by substituting the generically or specifically described reactants and/or operating conditions of this invention for those used in the proceding examples.

From the foregoing, description, one skilled in the art can easily ascertain the essential characteristics of this invention and, without departing from the spirit and scope thereof, can make various changes and modifications of the invention to adapt it to various usages and conditions.

| | | | CDK2 IC₅₀ | MCF-7 IC₅₀ |
|---|---|---|---|---|
| Example No. | R¹ | R³ | [μM] | [μM] |
| 1.0 | —Ph—OCH₃ | O | >5 | — |
| 1.1 | —Ph | O | 1 | — |
| 1.2 | —Ph—O—Si—(CH₃)₂C(CH₃)₃ | O | 0.15 | 4 |
| 1.3 | —Ph(CF₃)₂ | O | 0.6 | — |
| 1.4 | —Ph—Cl | O | 0.03 | 0.5 |
| 1.5 | —Ph—C(CH₃)₃ | O | 0.3 | |
| Compound No. 11, | —SO₃H | O | 0.3 | >10 |
| page 14, | | | | |
| Table 2 of | | | | |
| WO99/62503 | | | | |
| Compound No. 6, | —CH₃ | O | 3 | 1 |
| page 14, | | | | |
| Table 2 of | | | | |
| WO99/62503 | | | | |
| Compound No. 10, | H | NOH | 0.9 | 6 |
| page 14, | | | | |
| Table 2 of | | | | |
| WO99/62503 | | | | |

Ph = phenyl

It can be seen from the table that both in the enzyme test and in the cell test, the compounds according to the invention have significantly higher activities in the enzyme and in the MCF-7 cells than the compounds that are known from the closest prior art (WO99/62503). The compounds according to the invention are thus far superior to the known compounds. In this connection, it is to be noted that the substituent at R¹ is decisive to the superiority of the compounds according to the invention, and the other substituents do not produce any significant change in the effectiveness of the basic compounds.

1. Aryl-substituted indirubin derivatives of general formula I, 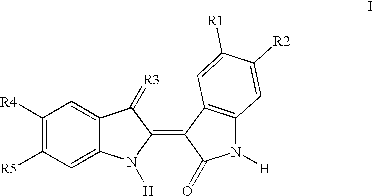
in which
R¹, R², R⁴ and R⁵ stand for hydrogen, hydroxy, C₁₋₆ alkoxy, aryloxy, trifluoromethyl, nitro, halogen, cyano or the group —SOₚR⁶, —SO₂NR⁷R⁸, or —COR⁹; or for aryl or heteroaryl that is optionally substituted by the same or a different component in one or more places with hydroxy, halogen, C₁-C₆ alkyl, amino, nitro, trifluoromethyl, —O—Si(C₁-C₆ alkyl)₃, cyano, COOC₁₋₄ alkyl, CO₁₋₄ alkyl, CO₁₋₄ aryl, SO₂(C₁₋₄ alkyl), SO₂ aryl and/or C₁-C₆ alkoxy,
R³ stands for oxygen or the group —NOR⁹, or
R¹ and R² or
R⁴ and R⁵ optionally form another C₃-C₆-membered ring,
R⁶ stands for hydrogen, for C₁-C₁₈ alkyl that is optionally substituted in one or more places with halogen, hydroxy and/or amino; aryl, heteroaryl or C₃₋₈ cycloalkyl that is optionally substituted in one or more places with halogen, hydroxy, amino, C₁-C₅ alkyl and/or C₁-C₆ alkoxy,
R⁷ and R8 are the same or different and stand for hydrogen or for C₁₋₁₈ alkyl, aryl, heteroaryl or acyl that is optionally substituted in one or more places with hydroxy, halogen and/or amino; or C₁-C₁₈ alkyl, C₃₋₈ cycloalkyl or C₃₋₈ cycloalkenyl that are optionally interrupted by one or more oxygen atoms, or
R⁷ or R⁸ together with the nitrogen atom of the amino group forms a C₃₋₈ cycloalkyl, which can contain one or more additional heteroatoms,
R⁹ stands for hydrogen or for C₁₋₆ alkyl that is optionally substituted in one or more places with hydroxy, halogen, C₁₋₄ alkoxy, amino, nitro, trifluoromethyl, cyano, COOC₁₋₄ alkyl, CO₁₋₄ alkyl, CO₁₋₄ aryl, SO₂(C₁₋₄ alkyl) and/or SO₂-aryl; or for aryl or heteroaryl that is optionally substituted in one or more places with halogen, C₁-C₆ alkyl, hydroxy, amino, nitro, trifluoromethyl, cyano, COOC₁₋₄ alkyl, CO₁₋₄ alkyl, CO₁₋₄ aryl, SO₂(C₁₋₄ alkyl), SO₂ arylamino and/or C₁-C₆ alkoxy, and
p is 0, 1 or 2,
whereby at least one of the two radicals R¹ and R² or radicals R⁴ and R⁵ means the aryl or hetaryl radical, as well as isomers and salts thereof.

2. Compounds of general formula I, according to claim 1, in which
R¹ stands for aryl or heteroaryl that is optionally substituted by the same or a different component in one or more places with halogen, hydroxy, C₁-C₆ alkyl, amino, nitro, trifluoromethyl, —O—Si(C₁-C₆ alkyl)₃, cyano, COOC₁₋₄ alkyl, CO₁₋₄ alkyl, CO₁₋₄ aryl, SO₂(C₁₋₄ alkyl), SO₂ aryl and/or C₁-C₆ alkoxy, and R² stands for hydrogen, hydroxy, C₁₋₆ alkoxy, aryloxy, trifluoromethyl, nitro, halogen, cyano or the group —SOₚR⁶, —SO₂NR⁷R⁸, or —COR⁹, or
R¹ stands for hydrogen, hydroxy, C₁₋₆ alkoxy, aryloxy, trifluoromethyl, nitro, halogen, cyano or the group —SOₚR⁶, —SO₂NR⁷R⁸, or —COR⁹, and
R² stands for aryl or heteroaryl that is optionally substituted in one or more places with halogen, hydroxy, C₁-C₆ alkyl, amino, nitro, trifluoromethyl, cyano, COOC₁₋₄ alkyl, CO₁₋₄ alkyl, CO₁₋₄ aryl, SO₂(C₁₋₄ alkyl), SO₂ aryl and/or C₁-C₆ alkoxy,
R⁴ and R⁵ stand for hydrogen, hydroxy, C₁₋₆ alkoxy, aryloxy, trifluoromethyl, nitro, halogen, cyano or the group —SOₚR⁶, —SO₂NR⁷R⁸, or —COR⁹; or for aryl or heteroaryl that is optionally substituted in one or more places with halogen, hydroxy, C₁-C₆ alkyl, amino, nitro, trifluoromethyl, cyano, COOC₁₋₄ alkyl, CO₁₋₄ alkyl, CO₁₋₄ aryl, SO₂ (C₁₋₄ alkyl), SO₂ aryl and/or C₁-C₆ alkoxy,
R³ stands for oxygen or the group —NOR⁹, or
R¹ and R² or
R⁴ and R⁵ optionally form another C₃-C₆-membered ring,
R⁶ stands for hydrogen or C₁₋₁₈ alkyl,
R⁷ and R⁸ are the same or different and stand for hydrogen or for C₁₋₁₈ alkyl, aryl, heteroaryl or acyl that is optionally substituted in one or more places with hydroxy, halogen and/or amino; or C₁₋₁₈ alkyl, C₃₋₈ cycloalkyl or C₃₋₈ cycloalkenyl that is optionally interrupted by one or more oxygen atoms, or
R⁷ or R⁸ together with the nitrogen atom of the amino group forms a C₃₋₈ cycloalkyl, which can contain one or more other heteroatoms,
R⁹ stands for hydrogen or for C₁₋₆ alkyl that is optionally substituted in one or more places with hydroxy, halogen, C₁₋₄ alkoxy, amino, nitro, trifluoromethyl, cyano, COOC₁₋₄ alkyl, CO₁₋₄ alkyl, CO₁₋₄ aryl, SO₂ (C₁₋₄ alkyl) and/or SO₂-aryl; or for aryl or heteroaryl that is optionally substituted in one or more places with halogen, C₁-C₆ alkyl, hydroxy, amino, nitro, trifluoromethyl, cyano, COOC₁₋₄ alkyl, CO₁₋₄ alkyl, CO₁₋₄ aryl, SO₂(C₁₋₄ alkyl), SO₂ arylamino and/or C₁-C₆ alkoxy, and
p is 0, 1 or 2, as well as isomers and salts thereof.

3. Compounds of general formula I, according to claims 1 and 2, in which
R¹ stands for phenyl, thiophenyl, furanyl, oxazolyl, thiazolyl, imidazolyl, pyridinyl, pyrimidinyl, triazinyl, quinolinyl or isoquinolinyl that is optionally substituted by the same or a different component in one or more places with halogen, hydroxy, C₁-C₆ alkyl, amino, nitro, trifluoromethyl, —O—Si(C₁-C₆ alkyl)₃, cyano, COOC₁₋₄ alkyl, CO₁₋₄ alkyl, CO₁₋₄ aryl, SO₂(C₁₋₄ alkyl), SO₂ aryl and/or C₁-C₆ alkoxy, and
R² stands for hydrogen, hydroxy, C₁₋₆ alkoxy, aryloxy, trifluoromethyl, nitro, halogen, cyano or the group —SOₚR⁶, —SO₂NR⁷R⁸, or —COR⁹, or
R¹ stands for hydrogen, hydroxy, C₁₋₆ alkoxy, aryloxy, —CF₃, nitro, halogen, cyano or the group —SOₚR⁶, —SO₂R⁷R⁸, or —COR⁹, and
R² stands for phenyl, thiophenyl, furanyl, oxazolyl, thiazolyl, imidazolyl, pyridinyl, pyrimidinyl, triazinyl, quinolinyl or isoquinolinyl that is optionally substituted in one or more places with halogen, hydroxy, C₁-C₆ alkyl, amino, nitro, trifluoromethyl, cyano, COOC₁₋₄ alkyl, CO₁₋₄ alkyl, CO₁₋₄ aryl, SO₂(C₁₋₄ alkyl), SO₂ aryl and/or C₁₋₆ alkoxy,
R⁴ and R⁵ stand for hydrogen, hydroxy, C₁₋₆ alkoxy, aryloxy, —CF₃, nitro, halogen, cyano or the group —SOₚR⁶, —SO₂NR⁷R⁸, or —COR⁹; or for aryl or heteroaryl that is optionally substituted in one or more places with halogen, hydroxy, C₁-C₆ alkyl, amino, nitro, trifluoromethyl, cyano, COOC₁₋₄ alkyl, CO₁₋₄ alkyl, CO₁₋₄ aryl, SO₂(C₁₋₄ alkyl), SO₂ aryl and/or C₁-C₆ alkoxy,
R³ stands for oxygen or the group —NOR⁹, or
R¹ and R² or
R⁴ and R⁵ optionally form another C₃-C₆-membered ring,
R⁶ stands for hydrogen or C₁₋₁₈ alkyl,
R⁷ and R⁸ are the same or different and stand for hydrogen or for C₁₋₁₈ alkyl, aryl, heteroaryl or acyl that is optionally substituted in one or more places with hydroxy, halogen and/or amino; or C₁₋₁₈ alkyl, C₃₋₈ cycloalkyl or C₃₋₈ cycloalkenyl that is optionally interrupted by one or more oxygen atoms, or
R⁷ or R⁸ together with the nitrogen atom of the amino group forms a C₃₋₈ cycloalkyl, which can contain one or more other heteroatoms,
R⁹ stands for hydrogen or for C₁₋₆ alkyl that is optionally substituted in one or more places with hydroxy, halogen, C₁₋₄ alkoxy, amino, nitro, trifluoromethyl, cyano, COOC₁₋₄ alkyl, CO₁₋₄ alkyl, CO₁₋₄ aryl, SO₂(C₁₋₄ alkyl) and/or SO₂ aryl; or for aryl or heteroaryl that is optionally substituted in one or more places with halogen, C₁-C₆ alkyl, hydroxy, amino, nitro, trifluoromethyl, cyano, COOC₁₋₄ alkyl, CO₁₋₄ alkyl, CO₁₋₄ aryl, SO₂(C₁₋₄ alkyl), SO₂ arylamino and/or C₁-C₆ alkoxy, and
p is 0, 1 or 2,
as well as isomers and salts thereof.

4. Compounds of general formula I, according to claims 1 to 3, in which
R¹ stands for phenyl or pyridyl that is optionally substituted by the same or a different component in one or more places with halogen, hydroxy, C₁-C₆ alkyl, amino, nitro, trifluoromethyl, —O—Si(C₁-C₆ alkyl)₃, cyano, COOC₁₋₄ alkyl, CO₁₋₄ alkyl, CO₁₋₄ aryl, SO₂(C₁₋₄ alkyl), SO₂ aryl and/or C₁-C₆ alkoxy, and
R² stands for hydrogen, hydroxy, C₁₋₆ alkyloxy, aryloxy, trifluoromethyl, nitro, halogen, cyano or the group —SOₚR⁶, —SO₂NR⁷R⁸, or —COR⁹, or
R¹ stands for hydrogen, hydroxy, C₁₋₆ alkoxy, aryloxy, trifluoromethyl, nitro, halogen, cyano or the group —SOₚR⁶, —SO₂NR⁷R⁸, or —COR⁹, and
R² stands for phenyl or pyridyl that is optionally substituted in one or more places with halogen, hydroxy, C₁-C₆ alkyl, amino, nitro, trifluoromethyl, cyano, COOC₁₋₄ alkyl, CO₁₋₄ alkyl, CO₁₋₄ aryl, SO₂(C₁₋₄ alkyl), SO₂ aryl and/or C₁-C₆ alkoxy,
R⁴ and R⁵ stand for hydrogen, hydroxy, C₁₋₆ alkoxy, aryloxy, —CF₃, nitro, halogen, cyano or the group —SOₚR⁶, —SO₂NR⁷R⁸ or —COR⁹; or for aryl or heteroaryl that is optionally substituted in one or more places with halogen, hydroxy, C₁-C₆ alkyl, amino, nitro, trifluoromethyl, cyano, COOC₁₋₄ alkyl, CO₁₋₄ alkyl, CO₁₋₄ aryl, SO₂(C₁₋₄ alkyl), SO₂ aryl and/or C₁-C₆ alkoxy,
R³ stands for oxygen or the group —NOR⁹, or
R¹ and R2 or
R⁴ and R⁵ optionally form another C₃-C₆-membered ring,
R⁶ stands for hydrogen or C₁₋₁₈ alkyl,
R⁷ and R⁸ are the same or different and stand for hydrogen or for C₁₋₁₈ alkyl, aryl, heteroaryl or acyl that is optionally substituted in one or more places with hydroxy, halogen and/or amino; or C₁₋₁₈ alkyl, C₃₋₈ cycloalkyl or C₃₋₈ cycloalkenyl that is optionally interrupted by one or more oxygen atoms, or
R⁷ or R8 together with the nitrogen atom of the amino group forms a C₃₋₈ cycloalkyl, which can contain one or more other heteroatoms,
R⁹ stands for hydrogen or for C₁₋₆ alkyl that is optionally substituted in one or more places with hydroxy, halogen, C₁₋₄ alkoxy, amino, nitro, trifluoromethyl, cyano, COOC₁₋₄ alkyl, CO₁₋₄ alkyl, CO₁₋₄ aryl, SO₂(C₁₋₄ alkyl) and/or SO₂-aryl; or for aryl or heteroaryl that is optionally substituted in one or more places with halogen, C₁-C₆ alkyl, hydroxy, amino, nitro, trifluoromethyl, cyano, COOC₁₋₄ alkyl, CO₁₋₄ alkyl, CO₁₋₄ aryl, SO₂(C₁₋₄ alkyl), SO₂ arylamino and/or C₁-C₆ alkoxy, and
p is 0, 1 or 2,
as well as isomers and salts thereof.

5. Compounds of general formula I, according to claims 1 to 4, in which
R¹ stands for phenyl or pyridyl that is optionally substituted by the same or a different component in one or more places with halogen, hydroxy, C₁-C₆ alkyl, amino, nitro, trifluoromethyl, —O—Si(C₁-C₆ alkyl)₃, cyano, COOC₁₋₄ alkyl, CO₁₋₄ alkyl, CO₁₋₄ aryl, SO₂(CO₁₋₄ alkyl), SO₂ aryl and/or C₁-C₆ alkoxy,
R² stands for hydrogen,
R⁴ and R⁵ stand for hydrogen, hydroxy, C₁₋₆ alkoxy, aryloxy, —CF₃, nitro, halogen, cyano, or the group —SOₚR⁶, —SO₂NR⁷R⁸, or —COR⁹; or for aryl or heteroaryl that are optionally substituted in one or more places with halogen, hydroxy, C₁-C₆ alkyl, C₁₋₆ alkoxy, amino, nitro, trifluoromethyl, cyano, COOC₁₋₄ alkyl, CO₁₋₄ alkyl, CO₁₋₄ aryl, SO₂(C₁₋₄ alkyl), SO₂ aryl and/or C₁-C₆ alkoxy,
R³ stands for oxygen or the group —NOR⁹, or
R⁴ and R⁵ optionally form another C₃-C₆-membered ring,
R⁶ stands for hydrogen or C₁₋₁₈ alkyl,
R⁷ and R⁸ are the same or different and stand for hydrogen or for C₁₋₁₈ alkyl, aryl, heteroaryl or acyl that is optionally substituted in one or more places with hydroxy, halogen and/or amino; or C₁₋₁₈ alkyl, C₃₋₈ cycloalkyl or C₃₋₈ cycloalkenyl that is optionally interrupted by one or more oxygen atoms, or
R⁷ or R⁸ together with the nitrogen atom of the amino group forms a C₃₋₈ cycloalkyl, which can contain one or more other heteroatoms,
R⁹ stands for hydrogen or for C₁₋₆ alkyl that is optionally substituted in one or more places with hydroxy, halogen, C₁₋₄ alkoxy, amino, nitro, trifluoromethyl, cyano, COOC₁₋₄ alkyl, CO₁₋₄ alkyl, CO₁₋₄ aryl, SO₂(C₁₋₄ alkyl) and/or SO₂-aryl; or for aryl or heteroaryl that is substituted in one or more places with halogen, C₁-C₆ alkyl, hydroxy, amino, nitro, trifluoromethyl, cyano, COOC₁₋₄ alkyl, CO₁₋₄ alkyl, CO₁₋₄ aryl, SO₂(C₁₋₄ alkyl), SO₂ arylamino and/or C₁-C₆ alkoxy, and
p is 0, 1 or 2,
as well as isomers and salts thereof.

6. Compounds of general formula I, according to claims 1 to 5, in which
R¹ stands for phenyl or pyridyl that is optionally substituted by the same or a different component in one or more places with halogen, hydroxy, C₁-C₆ alkyl, amino, nitro, trifluoromethyl, —O—Si(C₁-C₆ alkyl)₃, cyano, COOC₁₋₄ alkyl, CO₁₋₄ alkyl, CO₁₋₄ aryl, SO₂(C₁₋₄ alkyl), SO₂ aryl and/or C₁-C₆ alkoxy,
R² stands for hydrogen,
R⁴ and R⁵ stand for hydrogen, hydroxy, C₁₋₆ alkoxy, aryloxy, trifluoromethyl, nitro, halogen, cyano or the group —SOₚR⁶, —SO₂NR⁷R⁸, or —COR⁹; or for aryl or heteroaryl that is optionally substituted in one or more places with halogen, hydroxy, C₁-C₆ alkyl, amino, nitro, trifluoromethyl, cyano, COOC₁₋₄ alkyl, CO₁₋₄ alkyl, CO₁₋₄ aryl, SO₂(C₁₋₄ alkyl), SO₂ aryl and/or C₁-C₆ alkoxy,
R³ stands for oxygen or the group —NOR⁹, or
R⁴ and R⁵ optionally form another C₃-C₆-membered ring,
R⁶ stands for hydrogen or C₁₋₁₈ alkyl,
R⁷ and R⁸ are the same or different and stand for hydrogen or for C₁₋₈ alkyl, aryl, heteroaryl or acyl that is optionally substituted in one or more places with hydroxy, halogen and/or amino; or C₁₋₁₈ alkyl, C₃₋₈ cycloalkyl or C₃₋₈ cycloalkenyl that is optionally interrupted by one or more oxygen atoms, or
R⁷ or R⁸ together with the nitrogen atom of the amino group forms a C₃₋₈ cycloalkyl, which can contain one or more other heteroatoms,
R⁹ stands for hydrogen or for C₁₋₆ alkyl that is optionally substituted in one or more places with hydroxy, halogen, C₁₋₄ alkoxy, amino, nitro, trifluoromethyl, cyano, COOC₁₋₄ alkyl, CO₁₋₄ alkyl, CO₁₋₄ aryl, SO₂(C₁₋₄ alkyl) and/or SO₂ aryl; or for aryl or heteroaryl that is optionally substituted in one or more places with halogen, C₁-C₆ alkyl, hydroxy, amino, nitro, trifluoromethyl, cyano, COOC₁₋₄ alkyl, CO₁₋₄ alkyl, CO₁₋₄ aryl, SO₂(C₁₋₄ alkyl), SO₂-arylamino and/or C₁-C₆ alkoxy, and
p is 0, 1 or 2,
as well as isomers and salts thereof.

7. Compounds of general formula I, according to claims 1 to 6, in which
R¹ stands for phenyl that is optionally substituted by the same or a different component in one or more places with halogen, hydroxy, C₁-C₆ alkyl, amino, nitro, trifluoromethyl, —O—Si(C₁-C₆ alkyl)₃, cyano, COOC₁₋₄ alkyl, CO₁₋₄ alkyl, CO₁₋₄ aryl, SO₂(C₁₋₄ alkyl), SO₂ aryl and/or C₁-C₆ alkoxy,
R² stands for hydrogen,
R⁴ and R⁵ stand for hydrogen,
R³ stands for oxygen,
as well as isomers and salts thereof.

8. Compounds of general formula I, according to claims 1 to 7, in which
R¹ stands for phenyl that is optionally substituted by the same or a different component in one or more places with halogen, hydroxy, C₁-C₄ alkyl, amino, nitro, trifluoromethyl, cyano, —O—Si(C₁-C₆ alkyl)₃, or C₁-C₄ alkoxy,
R² stands for hydrogen,
R⁴ and R⁵ stand for hydrogen,
R³ stands for oxygen,
as well as isomers and salts thereof.

9. Use of the compounds of general formula I, according to claims 1 to 8, for the production of a pharmaceutical agent for treating cancer, auto-immune diseases, chemotherapy agent-induced alopecia and mucositis, cardiovascular diseases, infectious diseases, nephrological diseases, chronic and acute neurodegenerative diseases and viral infections.

10. Use according to claim 9, characterized in that cancer is defined as solid tumors and leukemia; auto-immune diseases are defined as psoriasis, alopecia and multiple sclerosis; cardiovascular diseases are defined as stenoses, arterioscleroses and restenoses; infectious diseases are defined as diseases that are caused by unicellular parasites; nephrological diseases are defined as glomerulonephritis; chronic neurodegenerative diseases are defined as Huntington's disease, amyotrophic lateral sclerosis, Parkinson's disease, AIDS dementia and Alzheimer's disease; acute neurodegenerative diseases are defined as ischemias of the brain and neurotraumas; and viral infections are defined as cytomegalic infections, herpes, hepatitis B and C, and HIV diseases.

11. Pharmaceutical agents that contain at least one compound according to claims 1 to 8.

12. Pharmaceutical agents according to claim 11 for treating cancer, auto-immune diseases, cardiovascular diseases, infectious diseases, nephrological diseases, neurodegenerative diseases and viral infections.

13. Compounds according to claims 1 to 8 and pharmaceutical agents according to claims 11 to 12 with suitable formulation substances and vehicles.

14. Use of the compounds of general formula I, according to claims 1 to 8, as inhibitors of cyclin-dependent kinases.

15. Use according to claim 14, wherein the kinase is CDK1, CDK2, CDK3, CDK4, CDK5, CDK6, CDK7, CDK8 or CDK9.

16. Use of the compounds of general formula I, according to claims 1 to 8, as inhibitors of the glycogen-synthase-kinase (GSK-3β).

17. Use of the compounds of general formula I, according to claims 1 to 8, in the form of a pharmaceutical preparation for enteral, parenteral and oral administration.
